SEQUENCE LISTING (2 sequences)

SEQ ID NO: 1 - DNA, 2172 bp, Artificial sequence
  Human Caco-2 cell
cactgcccaaggagaaagggctaattctctgcctatggagcaagttctgcagatggttccagagacgggagtcctgggcccagagccgagatgagcagaacctgctgcagcagaagaggatctgggagtctcctctccttctagctgccaaagataatgatgtccaggccctgaacaagttgctcaagtatgaggattgcaaggtgcaccagagaggagccatgggggaaacagcgctacacatagcagccctctatgacaacctggaggccgccatggtgctgatggaggctgccccggagctggtctttgagcccatgacatctgagctctatgagggtcagactgcactgcacatcgctgttgtgaaccagaacatgaacctggtgcgagccctgcttgcccgcagggccagtgtctctgccagagccacaggcactgccttccgccgtagtccctgcaacctcatctactttggggagcaccctttgtcctttgctgcctgtgtgaacagtgaggagatcgtgcggctgctcattgagcatggagctgacatccgggcccaggactccctgggaaacacagtgttacacatcctcatcctccagcccaacaaaacctttgcctgccagatgtacaacctgttgctgtcctacgacagacatggggaccacctgcagcccctggacctcgtgcccaatcaccagggtctcacccctttcaagctggctggagtggagggtaacactgtgatgtttcagcacctgatgcagaagcggaagcacacccagtggacgtatggaccactgacctcgactctctatgacctcacagagatcgactcctcaggggatgagcagtccctgctggaacttatcatcaccaccaagaagcgggaggctcgccagatcctggaccagacgccggtgaaagagctggtgagcctcaagtggaagcggtacgggcggccgtacttctgcatgctgggtgccatatatctgctgtacatcatctgcttcaccatgtgctgcatctaccgccccctcaagcccaggaccaataaccgcacgagcccccgggacaacaccctcttacagcagaagctacttcaggaagcctacatgacccctaaggacgatatccggctggtcggggagctggtgactgtcattggggctatcatcatcctgctggtagaggttccagacatcttcagaatgggggtcactcgcttctttggacagaccatccttgggggcccattccatgtcctcatcatcacctatgccttcatggtgctggtgaccatggtgatgcggctcatcagtgccagcggggaggtggtacccatgtcctttgcactcgtgctgggctggtgcaatgtcatgtacttcgcccgaggattccagatgctaggccccttcaccatcatgattcagaagatggtttttggcgacctgatgcgattctgctggctgatggctgtggtcatcctgggctttgcttcagccttctatatcatcttccagacagaggaccccgaggagctaggccacttctacgactaccccatggccctgttcagcaccttcgagctgttccttaccatcatcgatggcccagccaactacaacgtggacctgcccttcatgtacagcatcacctatgctgcctttgccatcatcgccacactgctcatgctcaacctcctcattgccatgatgggcgacactcactggcgagtggcccatgagcgggatgagctgtggagggcccagattgtggccaccacggtgatgctggagcggaagctgcctcgctgcctgtggcctcgctccgggatctgcggacgggagtatggcctgggagaccgctggttcctgcgggtggaagacaggcaagatctcaaccggcagcggatccaacgctacgcacaggccttccacacccggggctctgaggatttggacaaagactcagtggaaaaactagagctgggctgtcccttcagcccccacctgtcccttcctatgccctcagtgtctcgaagtacctcccgcagcagtgccaattgggaaaggcttcggcaagggaccctgaggagagacctgcgtgggataatcaacaggggtctggaggacggggagagctgggaatatcagatctgactgc

SEQ ID NO: 2 - protein, 721 aa, Artificial Sequence
  135, 174, 186, 199, 201, 229, 230, 444, 466, 587,590, 618, 623, 631: Human Caco-2 cell
LPKEKGLILCLWSKFCRWFQRRESWAQSRDEQNLLQQKRIWESPLLLAAKDNDVQALNKLLKYEDCKVHQRGAMGETALHIAALYDNLEAAMVLMEAAPELVFEPMTSELYEGQTALHIAVVNQNMNLVRAVLAXRASVSARATGTAFRSSPCNLIYFGEHPLSFAACVNSEEXVRLLIEHGDDIXAQDSLGNTVLHIXIXQPNKTFACQMYNLLLSYDRHGDHLQPLXXVPNHQGLTPFKLAGVEGNIVMFQHLMQKRKHTQWTYGPLTSTLYDLTEIDSSGDEQSLLELIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIILLVEVPDIFRMGVTRFFGQTILGGPFHVLIITYAFMVLVTMVMRLISAXGEVVPMSFALVLGWCNVMYFAXGFQMLGPFTIMIQKMVFGDLMRFCWLMAVVILGFASAFYIIFQTEDPEELGHFYDYPMALFSTFELFLTIIDGPANYNVDLPFMYSITYAAFAIIATLLMLNLLIAMMGDTHWRVAHERDXLWXAQIVATTVMLERKLPRCLWPRSGICGRXYGLGXRWFLRLEXRQDLNRQRIQRYAQAFHTRGSEDLDKDSVEKLELGCPFSPHLSLPMPSVSRSTSRSSANWERLRQGTLRRDLRGIINRGLEDGESWEYQI

(19) United States
(12) Patent Application Publication
(10) Pub. No.: US 2007/0037149 A1
(43) Pub. Date: Feb. 15, 2007

(54) Human calcium transporter 1 gene, screening method of calcium absorption regulating factor, and calcium absorption regulating factor

(76) Inventors: Makoto Shimizu, Tokyo (JP); Ryuichiro Sato, Tokyo (JP); Yoshihiko Takano, Saitama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/541,811

(22) PCT Filed: Aug. 25, 2003
(86) PCT No.: PCT/JP03/10691

§ 371 Date: Jul. 8, 2005

(30) Foreign Application Priority Data
Jan. 8, 2003 (JP) 2003001794

(51) Int. Cl. C12Q 1/68; C07H 21/04; C12P 21/06; G01N 33/20; C07K 14/705

(52) U.S. Cl. 435/6; 435/69.1; 435/320.1; 435/325; 530/350; 536/23.5; 436/80

(57) ABSTRACT

An object of the present invention is to provide helpful means which enables: genetic applications such as insertion into an expression vector and transformation of a human CaT1 gene; clarification of a calcium absorption activity mechanism such as the presence of a factor affecting the regulation of a calcium absorption activity of human CaT1; and confirmation and new finding of a calcium absorption regulator. Namely, a human CaT1 gene, a plasmid vector thereof, a transformant transformed by the plasmid vector thereof, a method of screening a calcium absorption regulator and a screening kit thereof, and a calcium absorption promoter are provided by the present invention.

### TECHNICAL FIELD

The present invention relates to human calcium transporter gene 1, a method of screening calcium absorption regulator, and a calcium absorption promoter obtained by the method.

### BACKGROUND ART

Calcium taken from food is absorbed by intestinal tract, mainly by small intestine.

Calcium absorption is performed by either one of the following two types depending on the condition of calcium supply. That is, when the calcium supply is sufficient, simple diffusion transport through an intracellular pathway in small intestinal epithelium is performed, while when the calcium supply is insufficient, active transport through cells is positively performed.

Of those, the active transport through the cells involves an epithelial calcium channel (ECaC) and a calcium transporter (CaT) on small intestinal brush border membrane as shown in FIG. 1. Genetic analysis and electrophysiological analysis indicate that both of them belong to the same channel family. The calcium channel is classified into calcium channel 1 (ECaC1) and calcium channel 2 (ECaC2). The calcium transporter is classified into calcium transporter 1 (CaT1) and calcium transporter 2 (CaT2). Recently, CaT1 and ECaC2 are thought to be the same.

It is elucidated that rabbit ECaC is expressed mainly in a duodenum located in the upper part of small intestine near the stomach and the calcium absorption takes place under a weakly acidic condition (see The Journal of Biological Chemistry 1999, Vol. 274, No.13, p8375-8378). Human ECaC1 is expressed mainly in kidney, small intestine, and pancreas and its gene has been identified (see Genomics 2000, 67, 48-53).

It is reported that rat CaT1 is expressed mainly in duodenum, jejunum, cecum, and the like and a reduction in calcium absorption activity under acidic conditions suggests a high possibility that the rat CaT1 participates in calcium absorption in a neutral environment in the upper part of the small intestine (see The Journal of Biological Chemistry 1999, Vol. 274, No.32, p22739-22746). Also, the gene of human CaT1 has been identified and it is known that existence of CaT1 is observed in the intestinal epithelium cells, its calcium absorption activity increases near neutrality, and various metal ions influence its activity (see Biochemical and Biophysical Research Communications 2000, Vol. 278, p326-332). However, there is no report on insertion of human CaT1 gene into an expression vector or on genetic applications including transformation. In addition, the calcium absorption activity mechanism, such as the presence of a factor affecting the regulation of the calcium absorption activity of human CaT1, has not been clarified yet.

Incidentally, to maintain the calcium homeostasis in the living body of an adult, generally 500 mg/day calcium is necessary. For this purpose, it is necessary that 300 mg/day calcium must be absorbed through the intestinal tract. To obtain this amount of calcium, 700 mg/day calcium including a nonabsorbed portion of calcium must be taken from food. Accordingly, food manufacturers have developed and put on market calcium-enriched foods, particularly milk, milk beverage, milk products, and the like. In this case, the calcium is added in the form of calcium phosphate, calcium carbonate, calcium lactate and the like. These calcium salts are solubilized and absorbed in acidic environments in the upper part of the small intestine. However, in neutral to basic environments in the lower part of the small intestine, most of the calcium salts are insolubilized and excreted without absorption.

However, calcium that is prevented from being insolubilized, if any, is positively absorbed through CaT1 and the like in the lower part of the small intestine.

On the other hand, in the case of aged persons, secretion of gastric acid becomes weak, and the acidic environment in the intestine shifts toward neutrality, leading to a decrease in the absorbability of calcium.

This also suggests importance of a study on the presence of a factor affecting the regulation of the calcium absorption activity of human CaT1, particularly one derived from food.

Calcium absorption promoters from the intestinal tract include sugars such as lactose, CPP (casein phosphopeptide), guar gum hydrolysates.

Calcium absorption promoting mechanism by lactose is considered to be interaction with the brush border membrane (Armbrecht, H. J., et al., J.Nutr., 106, 1976), but the details thereof are not clear. Another hypothesis is that since lactose is digested and absorbed more slowly compared with other sugars, it reaches the lower part of the intestinal tract, thus affecting the calcium absorption in the lower part of the intestinal tract (Allen, L. H., Am. J. Clin. Nutr., 35, 1982). CPP is considered to “trap calcium before the calcium is insolubilized (forms calcium phosphate, etc.) in neutral environment in the lower part of the intestinal tract to prevent insolubilization of the calcium, thereby increasing absorption through the intercellular pathway” (Naito, H., et al., J. Nutr. Sci. Vitaminol. (Tokyo)., 32, 1986). In any rate, the intestinal calcium channel has just been cloned; the factor regulating the calcium absorption might regulate the calcium absorption activity of the human CaT1 in some way, but no report supporting this is available.

Clarification of the relationship between the factor regulating calcium absorption and the calcium absorption activity of human CaT1 can increase the utility of the above-mentioned factor and can also pave the way to finding a new factor that regulates calcium absorption and using it as a component of medicines, nutritive supplements, food, or the like.

It is an object of the present invention to provide means which enables the genetic applications, such as insertion into an expression vector and transformation, of human CaT1 gene, clarifies the calcium absorption activity mechanism, such as the presence of a factor affecting the regulation of the calcium absorption activity of human CaT1, and contributes to the confirmation and new finding of a calcium absorption regulator, and a calcium absorption regulator obtained by the means.

### DISCLOSURE OF THE INVENTION

The inventors of the present invention made extensive studies to achieve the above-mentioned object and performed reverse transcriptase polymerase chain reaction and 5′ RACE PCR on RNA extracted from a human digestive tract cell. As a result, they were successful in obtaining an approximately full-length base sequence of human CaT1 gene. The human CaT1 gene was introduced into in a pMEHis vector to prepare a plasmid vector pMEHis-CaT1. This was used to be transformed into a CHO cell by a lipofection method to prepare a human CaT1 constant expression cell. Various food factors that affect calcium absorption were actually acted on the human CaT1 constant expression cell. As a result, it has been found that the various food factors affect the human CaT1 constant expression cell, that is, regulate the calcium absorption activity of human CaT1, thus achieving the present invention.

The present invention according to claim 1 is a human calcium transporter 1 gene containing a base sequence described in SEQ ID No: 1 in the sequence listing.

The present invention according to claim 2 is a plasmid vector containing the human calcium transporter 1 gene according to claim 1.

The present invention according to claim 3 is a transformant transformed with the plasmid vector according to claim 2.

The present invention according to claim 4 is a method of screening a factor that regulates calcium absorption, comprising confirming the amount of calcium incorporated by the transformant according to claim 3.

The present invention according to claim 5 is a kit for screening a factor that regulates calcium absorption, characterized by including the transformant according to claim 3.

The present invention according to claim 6 is a factor that promotes calcium absorption obtained by the method of screening according to claim 4.

The present invention according to claim 7 is a factor that promotes calcium absorption obtained by the kit for screening according to claim 5.

### BRIEF DESCRIPTION OF THE DRAWINGS

FIG. 1 is a schematic diagram illustrating calcium absorption by an intestinal tract (active transport through the inside of cell);

FIG. 2 is a diagram outlining cloning of human CaT1 gene;

FIG. 3 is a diagram showing the DNA sequence of human CaT1 gene and homology between human CaT1 and human ECaC;

FIG. 4 is a graph showing data of the transmembrane region of human CaT1;

FIG. 5 is a diagram showing the amino acid sequence of human CaT1 and homology between human CaT1 and rat CaT1;

FIG. 6 is a diagram showing an example of a plasmid vector according to claim 2 of the present invention;

FIG. 7 is a diagram showing an example of cultivation conditions of and means to confirm an increase in calcium uptake into cells of a transformant according to claim 3 of the present invention;

FIG. 8 is a diagram showing an example of means to confirm the amount of calcium incorporated into a transformant in a screening method according to claim 4 of the present invention;

FIG. 9 is a diagram showing an expression level of CaT1 mRNA in each digestive tract tissue site;

FIG. 10 shows results of Western blotting;

In FIG. 10, the right hand side shows results of CHO cells to which pMEHis-CaT1 was transfected while the left hand side shows results of CHO cells to which pMEHis-vector was transfected;

FIG. 11 is a graph showing a change, with the lapse of time, of ⁴⁵Ca²⁺ amount incorporated in cells;

In FIG. 11, “●” shows results of CHO cells to which pMEHis-CaT1 was transfected while “◯” shows results of CHO cells to which pMEHis-vector was transfected;

FIG. 12 is a graph showing the change, with the lapse of time, of the amount of calcium, with which human CaT1 gene involved to incorporate into cells, out of the total amount of calcium incorporated into the cell;

FIG. 13 is a graph illustrating the influence of pH on the uptake of ⁴⁵Ca²⁺;

FIG. 14 is a graph illustrating the influence of metal ions on the uptake of ⁴⁵Ca²⁺;

FIG. 15 is a graph illustrating the influence by food-derived factors on the uptake of ⁴⁵Ca²⁺;

FIG. 16 is a graph illustrating the influence by CWP-D on the uptake of ⁴⁵Ca²⁺;

FIG. 17 is a graph illustrating the influence of pretreatment time with CWP-D on the uptake of ⁴⁵Ca²⁺;

FIG. 18 is a graph illustrating the influence of CWP-D concentration on the uptake of ⁴⁵Ca²⁺;

FIG. 19 is a graph illustrating the influence of CWP-D on the uptake of ⁴⁵Ca²⁺ by Caco-2 cells;

FIG. 20 is a graph illustrating a calcium saturation curve in the presence or absence of CWP-D;

FIG. 21 is a Lineweaver-Bark plot;

FIG. 22 is a graph illustrating the influence of CWP-D (ODS adsorbing fraction) on the uptake of ⁴⁵Ca²⁺;

FIG. 23 is a graph illustrating results of FPLC and the influence of CWP-D (FPLC fraction) on the uptake of ⁴⁵Ca²⁺;

FIG. 24 is a graph illustrating results of HPLC;

FIG. 25 is a graph illustrating the influence of CWP-D components (peaks 1, 2, and 3) on the uptake of ⁴⁵Ca²⁺;

FIG. 26 is a graph illustrating purity of peak 1;

FIG. 27 is a graph illustrating the influence of synthetic peptide IPA on the uptake of ⁴⁵Ca²⁺; and

FIG. 28 is a graph illustrating the influence of synthetic peptide IPA, IPA analogue, and amino acids on the uptake of ⁴⁵Ca²⁺.

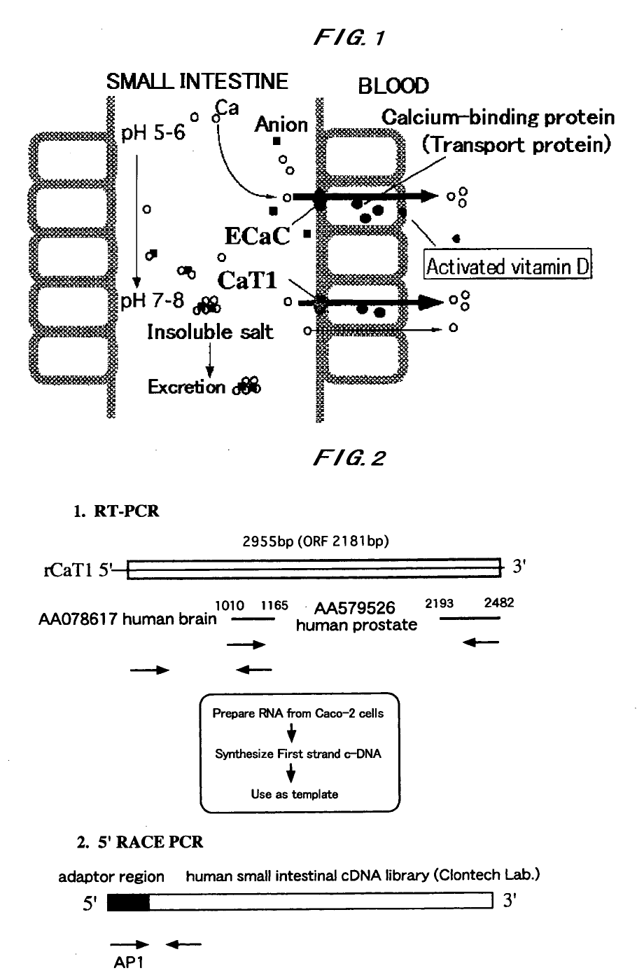

### BEST MODE FOR CARRYING OUT THE INVENTION

A human calcium transporter 1 gene according to claim 1 of the present invention is a human calcium transporter 1 gene containing a base sequence described in SEQ ID No: 1 in the sequence listing.

Most of the human CaT1 gene according to claim 1 of the present invention can be obtained by, with a use of total RNA or mRNA fraction of each tissue and cell of human digestive tract being prepared, directly subjecting them to Reverse Transcriptase Polymerase Chain Reaction (hereinafter, abbreviated as “RT-PCR”) to amplify them. However, there is a case in which 5′-end portion including an initiation codon is not elucidated only by RT-PCR. This portion can be obtained by further performing 5′ RACE (rapid amplification of cDNA end) PCR.

A series of operations can be performed, for example, according to the outline shown in FIG. 2.

Each tissue and cell of human digestive tract that can be used is not particularly limited and includes tissues and cells such as those of esophagus, stomach, duodenum, ileum, jejunum, ascending colon, descending colon, transverse colon, cecum, rectum, and liver. For example, Caco-2 cell, which is an example of human small intestine epithelium cells, is available from American Type Culture Collection and the like. Preparation of RNA from each tissue and cell of human digestive tract, for example in the case of the Caco-2 cell, can be performed by the methods in the examples described later on.

According to the RT-PCR method, DNA is obtained from RNA by using a reverse transcriptase and the obtained DNA is amplified by PCR. The method is preferable because it can be operated conveniently and advantageously by using a kit such as First strand cDNA synthesis kit (manufactured by Pharmacia biotech) under the conditions recommended for the kit.

A primer that is used in PCR of a cDNA obtained by a reverse transcription reaction can be designed based on, for example, sequence information on a rat CaT1 and sequence information on a human gene that has a high homology with a rat CaT1 according to data on EST (BLAST, etc.).

Reaction liquid and cycle of PCR can be performed under conditions that are usually adopted. It is preferable that annealing temperature be set depending on the designed primer and elongation time be set depending on the target fragment size.

5′ RACE PCR is preferable to be proceed conveniently and advantageously by using a kit such as human small intestine Marathon-Ready (trademark) cDNA (manufactured by Clonetech) according to the manual recommended for the kit used.

The design of the primer that can be used include one that is designed based on the information on the C-end of the cDNA of the human CaT1 gene obtained by the previously described RT-PCR and information on the part near initiation codon of the cDNA of the rat CaT1 gene (see The Journal of Biological Chemistry 1999, Vol. 274, No. 13, p8375-8378), and an adapter primer attached to the human small intestine Marathon-Ready (trademark) CDNA (manufactured by Clonetech).

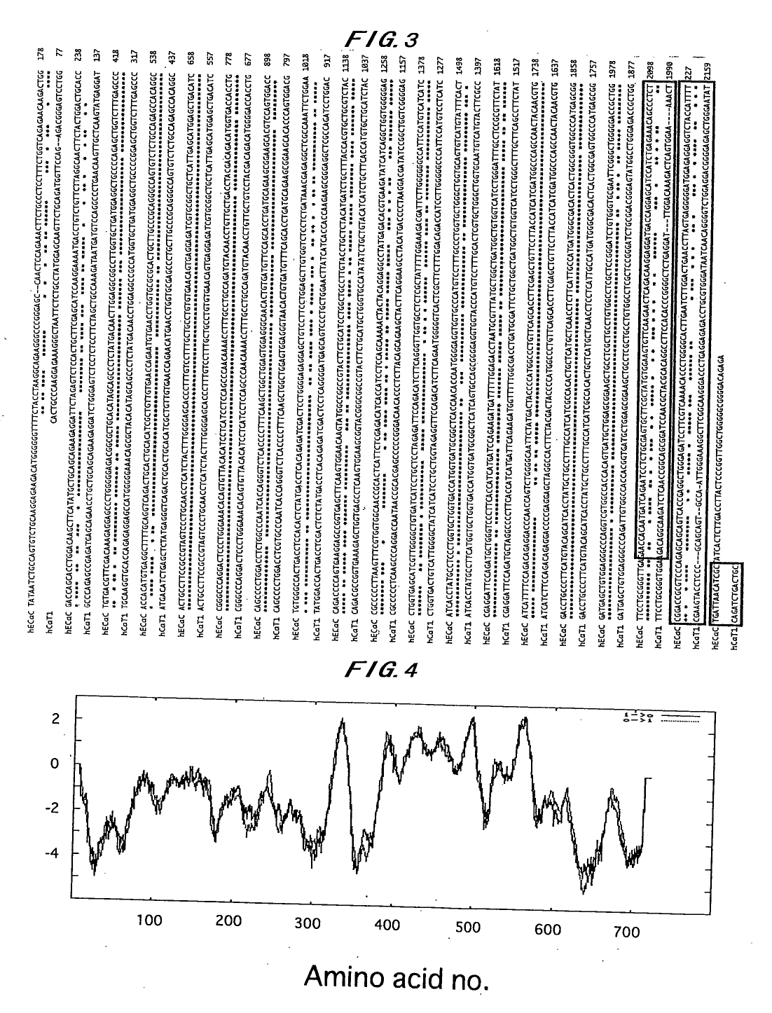

The human CaT1 gene according to claim 1 of the present invention contains the base sequence described in SEQ ID No: 1 in the sequence listing. The human CaT1 gene according to claim 1 of the present invention has about 85% homology with the rat CaT1 gene on a base level and as shown in FIG. 3, about 85% homology with a human ECaC gene recently reported (see lower part of FIG. 3; see Genomics 2000, 67, 48-53), however, a low homology of about 50% in a region of about 300 bp from the C-end.

Alternatively, the human CaT1 gene according to claim 1 of the present invention contains the amino acid sequence described in SEQ ID NO: 2 in the sequence listing.

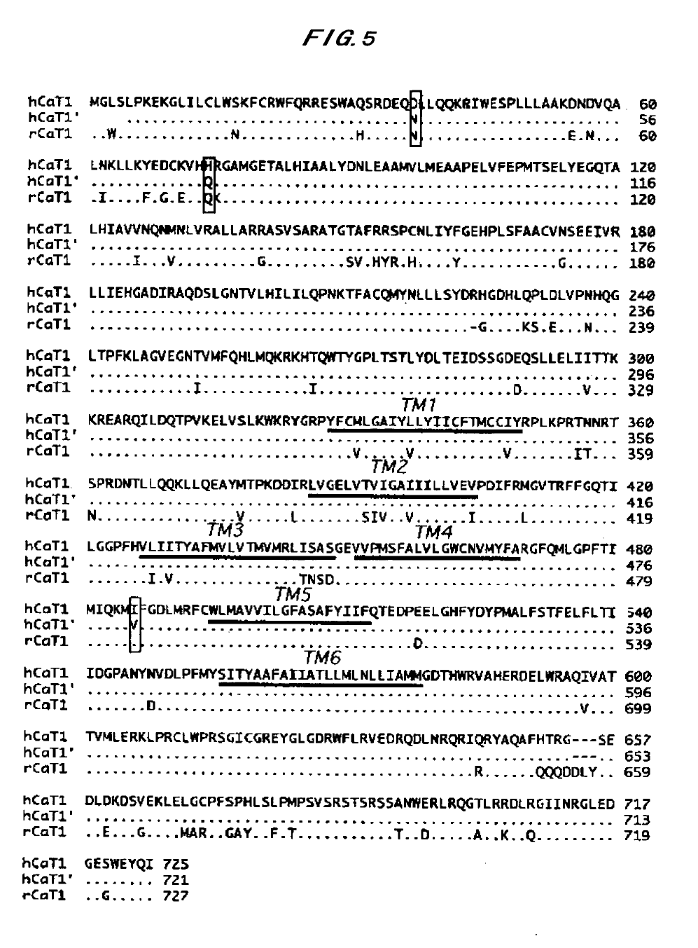

Conformation of the human CaT1 gene on an amino acid level is presumed to be of a 6-time transmembrane type as shown in FIG. 4 and have a pore region between the fifth and sixth transmembrane sites. In addition, the presumption of the conformation can be performed utilizing websites such as http://sosui.proteome.bio. tuat.ac.jp/cgi-bin/sosui.cgi?/sosui_submit.html.

As shown in FIG. 5, the amino acid sequence described in SEQ ID No: 2 in the sequence listing have only three different amino acids as compared with the amino acid sequence of the human CaT1 gene (see middle part of FIG. 5) reported by Hediger, et al. (see Biochemical and Biophysical Research Communications 2000, Vol. 278, p326-332). In addition, the amino acid sequence described in SEQ ID No: 2 has two parts in an intracellular region on an N-end that are common with the cDNA of the rat CaT1 gene (see the lower part of FIG. 5; see The Journal of Biological Chemistry 1999, Vol. 274, No. 13, p8375-8378).

The human CaT1 gene according to claim 1 of the present invention is not limited particularly to those obtained by the method described above as far as it contains the base sequence described in SEQ ID No: 1 and can be obtained by amplification using a synthetic primer that has a part of the base sequence coding the human CaT1 gene according to claim 1 of the present invention by the PCR method, or by hybridization of DNA incorporated in an appropriate vector with a part or the entire region of the human calcium transporter 1 gene according to claim 1 of the present invention. The hybridization method can be performed according to a method described in, for example, Molecular Cloning, 2nd ed. (J. Sambrook, et al., Cold Spring Harbor Lab. Press, 1989).

The human CaT1 gene according to claim 1 of the present invention can be ligated to an appropriate expression vector directly or after digestion with a restriction enzyme or attachment of a linker as desired, depending on the purpose. The present invention according to claim 2 provides such a plasmid vector.

In other words, according to claim 2 of the present invention, there is provided a plasmid vector containing a human calcium transporter 1 gene according to claim 1.

The plasmid vector of the present invention according to claim 2 can be produced by, for example, cleaving a DNA fragment from the human CaT1 gene according to claim 1 of the present invention and ligating the DNA fragment to the downstream of a promoter in an appropriate expression vector.

The expression vectors that can be used include those expression vectors derived from Escherichia coli, Bacillus subtilis, and yeast. Specifically, for example, a pMEHis vector can be used.

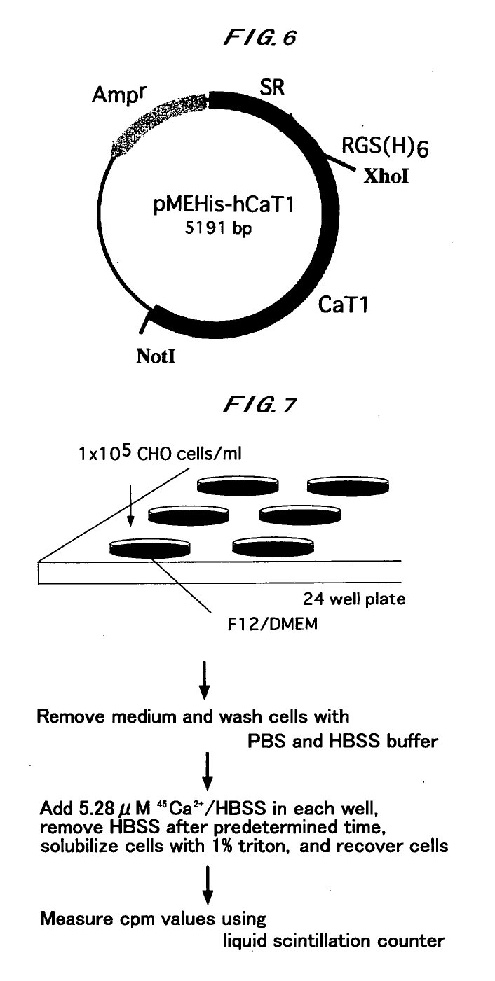

When the pMEHis vector is used, a primer is designed to enable introduction of Xho I and Not I sites into a human CaT1 gene and after RT-PCR is performed, the primer is inserted into the Xho I and Not I sites in a multicloning site of the pMEHis vector to obtain a plasmid vector pMEHis-CaT1 as shown in FIG. 6.

Using the thus-constructed plasmid vector according to claim 2 of the present invention, a transformant can be produced. According to claim 3 of the present invention, there is provided such a transformant.

In other word, according to claim 3 of the present invention, there is provided a transformant transformed with a plasmid vector according to claim 2.

According to claim 3 of the present invention, target hosts that can be used for transformation include Escherichia bacteria, Bacillus bacteria, yeasts, insect cells, insects, and animal cells. Of those, CHO (Chinese Hamster Ovary) cells are preferable. To transform (transfect) a CHO cell, for example, a lipofection method can be used. A transfection by the lipofection method is conveniently and advantageously performed by using a kit such as LIPOFECTAMIN Reagent kit (manufactured by GIBCO BRL), and thus is preferable.

The transformant according to claim 3 of the present invention is a transformant that is transformed with the plasmid vector according to claim 2 and in other words, a transformant that is transformed with a plasmid vector that contains the human calcium transporter 1 gene.

To confirm that the transformant according to claim 3 of the present invention contains the human CaT1 gene and the human CaT1 gene is expressed (that is, as a result of the gene expression in the cell, a human calcium transporter 1 is contained), for example, Western blotting using an anti-hCaT1 antibody prepared in a rabbit can be used. Specifically, in the case of, for example, a CHO cell used as a host, a colony of the transfected CHO cells is selected using blasticidin and solubilized with an appropriate nonionic surfactant (for example, 1% NP-40 (Nonidet P-40 surfactant, manufactured by Nacalai Tesque, Inc.). After purification, the remaining cells are removed by SDS-PAGE or the like, the supernatant is collected, and proteins contained in the cells are extracted. Meanwhile, a construct having a part of base sequences of the human CaT1 gene inserted in an appropriate expression vector is prepared. This construct is transfected to Escherichia coli to obtain a fusion protein. The fusion protein is used as an antigen of an anti-human CaT1 antibody prepared in a rabbit, and Western blotting is performed. When a specific band is observed, it is apparent that the human CaT1 gene is contained and expressed.

The nonionic surfactant that can be used for solubilization of cells is preferably the above-mentioned NP-40. However, more popular surfactants such as triton X100 can also be used.

Cultivation of the transformant according to claim 3 of the present invention in an appropriate condition allows the transformant to serve as a human CaT1 constant expression cell and express the human calcium transporter 1 gene, whereby increasing calcium uptake into the cell. An example of the cultivation of the transformant is performed as follows. In the case of CHO cell as a host as shown in FIG. 7, the transformant cultivated in a medium such as DMEM/F12 medium (for example, DMEM/F12 (1:1) Medium , Base Catalog No. 11320, GIBCO (trademark) manufactured by Invitrogen Corporation) at 35 to 40° C., preferably in a CO₂ environment is removed of the medium and washed with a buffer, such as PBS (Phosphate-Buffered Saline) buffer (Amer. J. of Cancer (1936) 27: 55), and HBSS (Hanks' Balanced Salt Solutions) buffer (Hanks, J. H. and Wallace, R. E., Proc. Soc. Exp. Biol. Med. (1949) 71, 196. Modification-National Institutes of Health). Then, a buffer containing calcium labeled with radioactive isotope (for example, ⁴⁵Ca²⁺) is added and after a predetermined time, the added buffer was removed and the cells were solubilized with a surfactant. The transformant is recovered and the amount of ⁴⁵Ca²⁺ incorporated into the cells is measured by using a liquid scintillation counter or the like to confirm an increase in calcium uptake into the cells.

The effects of pH and metal ions on the calcium absorption can be examined by confirming the amount of calcium incorporated into the transformant when pH is changed or various metal ions are acted on the transformant according to claim 3 of the present invention.

Factors that regulate calcium absorption can be screened by confirming the amount of calcium incorporated into the transformant when samples such as peptides, proteins, and sugars derived from microorganisms, animals and plants, and foods are acted on the transformant according to claim 3 of the present invention. According to claim 4 of the present invention, there is provided such a screening method.

In other words, according to claim 4 of the present invention, there is provided a method of screening a factor that regulates calcium absorption, characterized by confirming an amount of calcium incorporated into a transformant according to claim 3.

Specifically, the screening method according to claim 4 of the present invention determines the calcium absorption regulating activity of a sample by measuring the amount of incorporated calcium labeled with a radioactive isotope (for example, ⁴⁵Ca²⁺) and comparing the result as an index for calcium absorption regulating activity, thereby confirming the amount of the calcium incorporated into the transformant.

The factors that regulate the calcium absorption include compounds and salts thereof that promote or inhibit the calcium absorption in each tissue and cell of human digestive tracts.

Examples of the samples include peptides, proteins, and sugars derived from microorganisms, animals and plants, and foods. Those compounds may be either novel compounds or known compounds. In particular, use of peptides, proteins, sugars, and the like derived from foods as samples is preferable since it can increase utility as components of medicines, nutritive supplements, and foods.

The screening method according to claim 4 of the present invention is to confirm the amount of calcium incorporated into the transformant according to claim 3. First, preferably, the transformant according to claim 3 is washed, when needed, with an appropriate buffer and then solubilized or suspended in a surfactant. The buffer may be any buffer that does not inhibit the calcium absorption activity of the human CaT1 of the present invention, preferably buffers such as phosphate buffer and HBSS buffer having a pH of about 7 to 8. As a surfactant, the above-mentioned surfactants can be used.

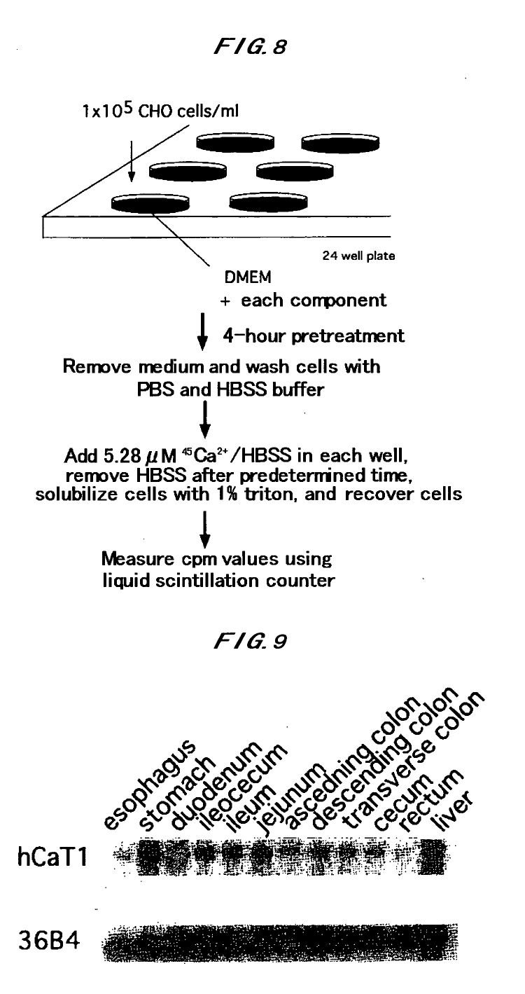

Confirmation of the amount of the calcium incorporated into the transformant according to claim 3 of the present invention is performed, for example, as shown in FIG. 8. That is, a medium that contains the above-mentioned sample is added to the transformant rendered in a state suitable for screening as described above and pretreatment is performed for 4 hours. Thereafter, the medium is removed, the transformant is washed with a PBS, HBSS buffer or the like, and a buffer containing ⁴⁵Ca²⁺ is added and removed the buffer after a lapse of predetermined time. After the cells are solubilized with a surfactant, the transformant is recovered and the amount of ⁴⁵Ca²⁺ incorporated into the cell is measured by a liquid scintillation counter or the like. As a control, measurement of the amount of calcium is performed in the same manner except that the sample is not added. As a standard of screening of factors that regulate calcium absorption, for example, results of both the sample and the control are subjected to statistic treatment such as ANOVA and used: when the significance level is less than 0.05 (n=3), the sample can be screened as a factor that promotes calcium absorption; and in other cases, the sample can be screened as a factor that inhibits calcium absorption.

The present invention according to claim 5 provides a kit for conveniently performing the screening method according to claim 4 of the present invention.

That is, the present invention according to claim 5, there is provided a kit for screening a factor that regulates calcium absorption containing the transformant according to claim 3.

Use of the screening method according to claim 4 of the present invention and the kit for screening according to claim 5 of the present invention enables to obtain a factor that regulates calcium absorption, particularly a factor that promotes calcium absorption with ease. The present invention according to claims 6 and 7 provides such a calcium absorption regulator.

The present invention according to claim 6 may be a factor that promotes calcium absorption obtained by the method of screening according to claim 4.

The present invention according to claim 7 may be a factor that promotes calcium absorption obtained using the kit for screening according to claim 5.

The factors that promote calcium absorption according to claims 6 and 7 of the present invention include a peptide consisting of three amino acids, such as Ile-Pro-Ala (IPA). Such a peptide IPA can be obtained by subjecting an enzymatic degradation product of cheese whey (CWP-D) that is obtained by digesting cheese whey with a proteolytic enzyme such as protease A to various purification means such as ODS column chromatography, FPLC, and HPLC, and performing the screening method according to claim 4 of the present invention or using the kit for screening according to claim 5 of the present invention in each purification stage to arbitrarily screen factors that inhibit calcium absorption. In addition, the factors that promote calcium absorption according to claims 6 and 7 of the present invention can be obtained by synthesizing a peptide consisting of the above-mentioned sequence Ile-Pro-Ala.

The factors that promote calcium absorption according to claims 6 and 7 of the present invention may be natural substances derived from components in foods, animals and plants, as well as synthetic substances. The molecular weight and protein structure of the calcium absorption promoters are not limited particularly. Specific examples thereof include CWP-D and its purified products.

The factors that promote calcium absorption according to claims 6 and 7 of the present invention can be used as a component of medicines, nutritive supplements, foods, and the like to increase their utility, and at the same time are useful as a substance that paves the way to utilization of a component of medicines, nutritive supplements, foods, and the like, serving as a new factor that regulates calcium absorption.

Hereinafter, the present invention is explained in more detail by way of examples.

### EXAMPLE 1

(1) Cloning of Human CaT1 Gene

First, human CaT1 gene cloning was performed. That is, according to the outline as shown in FIG. 2, most part of the DNA of the human CaT1 gene was cloned by RT-PCR (see 1. in FIG. 2) and then 5′ RACE PCR was performed for the 5′-end portion (see 2. in FIG. 2).

1. RT-PCR (see 1.in FIG. 2)

RNA was prepared from a Caco-2 cell, one of human small intestinal epithelium cells (obtained from American Type Culture Collection), and reverse transcription reaction was performed based on this to synthesize a first strand cDNA.

Preparation of RNA from a Caco-2 cell is as follows.

The Caco-2 cell was cultured using a DMEM medium (Manufactured by GIBCO™ Invitrogen Corporation, Base Catalog No. 11965: CaCl₂ (anhyd.) 200.00 mg/l, KCl 400.00 mg/l, NaCl 6400.00 mg/l, NaHCO₃ 3700.00 mg/l, NaH₂PO₄.H₂O 125.00 mg/l, D-glucose 4500.00 mg/l, L-glutamin 584.00 mg/l, L-isoleucine 105.00 mg/l, L-leucine 105.00 mg/l, L-lysine.HCl 146.00 mg/l) containing 10% FCS, 4 mM L-glutamin, 50IU/ml penicillin, and 50 μg/ml of streptomycin and under a wetting condition of 37° C. and 5% CO₂ in a 100 mm diameter dish, thereby a confluent cell was obtained after 3 to 4 days culture.

The medium was removed and ISOGEN in an amount of 1 ml per 100 mm-diameter dish was added to the remaining cells and the cells were scraped with a rubber policeman to dissolve the cells and the resultant was recovered in a 1-ml tube. After the resultant was homogenized with a 1-ml syringe with a 25G needle a few times, 200 μl of chloroform was added and the mixture was vortexed and centrifuged at 15,000 rpm for 15 minutes at 4° C. The supernatant was transferred into another tube, and 500 μl of isopropanol was added thereto and mixed and diluted well. The resultant mixture was left to stand at room temperature for 5 to 10 minutes and then centrifuged at 15,000 rpm for 10 minutes at 4° C. The supernatant was removed, the precipitate was washed with 75% ethanol and air-dried, and dissolved in an appropriate amount of RNase-free water. The sample was stored at −80° C.

The reverse transcription reaction was performed using First strand cDNA synthesis kit (manufactured by Pharmacia Biotech). That is, 1 to 5 μg of total RNA prepared from the Caco-2 cells was taken in a 1-ml tube. After the cells were dried in a vacuum centrifuge, the cells were dissolved in 8 μl of RNase-free water and heat-treated at 65° C. for 10 minutes. The resultant was immediately ice-cooled. Then, added to the resultant were 1 μl of Pd(N)₆ primer, 1 μl of DTT solution, and 5 μl of Bulk First-Strand cDNA Reaction Mix, respectively. After the mixture was messed up to 15 μl, it was incubated at 37° C. for 1 hour. Then, the mixture was heated at 65° C. for 15 minutes and then immediately ice-cooled. The sample was stored at −20° C.

PCR was performed using this cDNA as a template. A sense primer and an antisense primer designed based on the information of sequence on the rat CaT1 and the information on the human gene that has a high homology with rat CaT1 (AA078617 human brain and AA579526 human prostate) from the EST database (http://www.Evolution.bio.titech.ac.jp/keyword/est.html) were used. The homology analysis was performed on the database site (see (BLAST) http://www.Genome.ad.jp/japanese/(http://www. Hulinks.co.jp/software/turboblast/).

PCR was performed by a series of heat treatments of 94° C. for 2 to 3 minutes, 94° C. for 30 seconds, Tm° C. for 30 seconds, and 72° C. for XX (minutes or seconds) each repeated for 30 cycles using Accu Taq-LA polymerase PCRbuffer (10×), Accu Taq-Labuffer (10×), Deoxynucleotide mix, and Dimethyl Sulfoxide (all manufactured by SIGMA). The annealing temperature (Tm) was determined depending on the designed primer and the elongation time (XX (minutes or seconds) was determined depending on the target fragment size. 2. Cloning of 5′-end region containing unknown initiation codon (see 2. in FIG. 2)

Human small intestinal cDNA library manufactured by Clonetech lab. was subjected to 5′ RACE PCR to clone a 5′-end region containing unknown initiation codon.

The 5′ RACE PCR was performed using human small intestine Marathon-Ready (trademark) CDNA (manufactured by Clonetech lab.). The primer used was one that is designed based on the information on the C-end of the cDNA of the human CaT1 gene obtained by the previously described RT-PCR and the information on the part near initiation codon of the cDNA of the rat CaT1 gene (see The Journal of Biological Chemistry 1999, Vol. 274, No. 13, p8375-8378), and the one that is an adapter primer attached to the human small intestine Marathon-Ready (trademark) cDNA (manufactured by Clontech lab.).

As a result, substantially full-length of the human CaT1 gene except for four bases including the initiation codon was successfully cloned. The substantially full-length base sequence of the human CaT1 gene is as described in SEQ ID NO: 1 in the sequence listing. On a base level, this had a homology of about 85% with the rat CaT1 gene. As shown in “DNA sequence of Human CaT1 and Homology with human ECaC” in FIG. 3, it has been confirmed that this had a homology of about 85% with recently reported human ECaC gene (see the lower part of FIG. 3; see Genomics 2000, 67, 48-53), however, it had a low homology of about 50% in a region of about 300 bp from the C-end.

The (substantially) full-length amino acid sequence of the human CaT1 gene is as described in SEQ ID No: 2 in the sequence listing.

Presumption of the conformation on an amino acid level from the amino acid sequence of the human CaT1 gene referring to the website of http://sosuiproteome.bio.tuat.ac.jp/cgi-bin/sosui. cgi?/sosui submit.html suggests that the human CaT1 is a 6-transmembrane type and has a pore region between the fifth and sixth transmembrane sites as shown in FIG. 4. Comparison of the amino acid sequence of the human CaT1 gene with the amino acid sequence of the human CaT1 gene (see middle part of FIG. 5) reported by Hediger, et al., (Biochemical and Biophysical Research Communications 2000, Vol. 278, p326-332) indicates that only three amino acids are different as shown in FIG. 5. In addition, the amino acid sequence of the human CaT1 gene has two parts in the intracellular region on the N-end that are identical with the cDNA of the rat CaT1 gene (see the lower part of FIG. 5; see The Journal of Biological Chemistry 1999, Vol. 274, No.13, p8375-8378).

(2) Confirmation of Expression Level of Human CaT1 Gene

Then, the expression level of the human CaT1 gene at each tissue site of human digestive tract (esophagus, stomach, duodenum, ilenium, jejunum, ascending colon, descending colon, transverse colon, cecum, rectum, and liver) was confirmed by performing Northern blotting in the following procedure.

1. Prehybridization

The membrane used is a membrane of human digestive system MTN Blot (Clonetech). In this membrane, RNA from each digestive organ has previously been transferred and fixed thereon.

A heat-dried membrane was immersed in 2×SSC and placed in a HybriPack, to which was added a prehybridization solution (a mixture of 10 μl salmon testes DNA treated at 95° C. for 10 minutes and rapidly cooled in ice and 0.5 ml of a hybrydization solution), and the HybriPack was sealed with a sealer so that no bubbles are formed on the membrane. The sealed HybriPack was left to stand in a water bath at 42° C. for 3 hours or more.

Note that the composition of the hybridyzation solution is as follows.

[Composition of hybridyzation solution]

| (final) | (stock solution) |
|---|---|
| 5× Denhardt's | 50× |
| 5× SSPE (4 times diluted 20× SSPE shown below) | 20× |
| 50% formamide | |
| 0.1% SDS | 10% |
| 20× SSPE | |
| NaCl | 175.3 g |
| NaH₂PO₄.H₂O | 7.6 g |
| EDTA.2Na | 7.4 g |
| milliQ water | up to 1 L |

(The whole was adjusted to a pH at 7.4 with NaOH and autoclaved)
2. Preparation and Purification of Probe

The preparation and purification of a probe were performed using a megaprime labelling system (manufactured by Amersham Pharmacia). That is, about 25 ng of template DNA was made 14 μl with milliQ water and heat-treated at 95° C. for 5 minutes, and then rapidly cooled in ice. Added thereto were 5 μl of a primer solution, 2.5 μl of a labeling buffer, 2.5 μl of α-32P-dCTP, and 1 μl of enzyme. The mixture was left to stand in a water bath at 37° C. for 1 to 2 hours. Thereafter, 24 μl of 0.2% SDS/TE and 1 μl of 0.5 M EDTA were added thereto to obtain a nonpurified probe purification liquid. The column for purifying a probe was prepared by stuffing silicone-treated glass wool on the apex of a blue chip, which was placed in a 1.5-ml microtube and then 1 ml of Sephadex G-50 (washed with TE and then equilibrated and autoclaved) was introduced in the chip.

The nonpurified probe liquid was added to the column for purifying the probe. When the nonpurified probe liquid moved into the resin, an appropriate amount of TE was added to the column, and eluate was recovered in the 1.5 ml microtube sequentially in a rate of 150 to 200 μl/fraction. The recovered fraction was measured for radiant quantity of ³²P by a liquid scintillation counter. The fraction corresponding to a first peak was used as a probe liquid.

3. Hybridization

The probe liquid was introduced into a 1.5-ml tube to make about 2,500,000 cpm. 5 μl of salmon testes DNA was added thereto and the mixture was heat-treated at 95° C. for 10 minutes and rapidly cooled in ice. Then, 0.5 ml of a hybridyzation solution was added thereto to obtain a hybridization solution. The membrane subjected to the prehybridization in 1. described above was introduced in a new HybriPack and the total amount of the hybridization solution was introduced therein. The Hybripack was sealed with a sealer so that no bubbles were formed on the membrane and left to stand in a water bath at 42° C. overnight.

4. Cleaning and Exposure to Light of Membrane

The membrane was lightly rinsed in a Tupperware with water and then washed with 2×SSC and 0.1% SDS at 37° C. for 2 to 3 minutes for the first time and for 20 to 30 minutes for the second time. Then, the membrane was washed with 0.1×SSC and 0.1% SDS twice each for 20 minutes. After a decrease in the background radioactivity was confirmed on a survey meter, the moisture of the membrane was removed with paper towel and the membrane was fixed to filter paper and wrapped in wrap. The wrapped membrane was placed in a holder for exposure and was exposed to light on an imaging plate (IP) for an appropriate time. The exposed IP was transferred to a special-purpose magazine to scan and analyze it by BAS2000 manufactured by FUJI FILM.

FIG. 9 shows expression levels of mRNA of the human CaT1 gene at each tissue site of the digestive tract. The lower part of FIG. 9 shows results obtained by repeating the same procedure as the above-mentioned procedure except that human cDNA (36B4) as a control was used instead of the human CaT1 gene.

As a result, as apparent from FIG. 9, expression of human CaT1 gene at each tissue site of the digestive tract was observed. In particular, the expression of the human CaT1 gene was widely confirmed from the stomach and the upper part to the lower part of the small intestine. This suggests that also in humans, calcium uptake in the lower part of small intestine in the neutral environment is performed through the human CaT1.

(3) Preparation of Human CaT1 Constant Expression Cells

Because of insufficient reports on the regulation mechanism of calcium uptake through the human CaT1, the inventors of the present invention prepared a vector that incorporated therein the human CaT1 gene as shown in FIG. 6 and transformed into CHO cell with the prepared vector to prepare human CaT1 constant expression cells to try to elucidate the regulating mechanism.

First, human CaT1 gene was inserted into Xho I and Not I sites of the pMEHis vector. That is, a primer was designed to enable introduction of Xho I and Not I sites into the human CaT1 gene and after RT-PCR was performed, the amplified human CaT1 gene were inserted into the Xho I and Not I sites in the multicloning site of the pMEHis vector to obtain a plasmid vector pMEHis-CaT1. This was transfected to CHO (Chinese hamster ovary) cell that does not express human CaT1 gene inherently by a lipofection method.

The transfection by the lipofection method was performed using LIPOFECTAMIN Reagent kit (manufactured by GIBCO BRL). That is, firstly 2 to 10 μg of plasmid DNA and 8 μl of plus reagent were introduced in 250 μl of DMEM/F12 medium and the mixture was stirred lightly and left to stand at 37° C. for 15 minutes. Thereafter, 250 μl of separately provided DMEM/F12 medium containing 12 μl of lipofectamin was added thereto. After the mixture was lightly stirred, the mixture was left to stand at 37° C. for further 15 minutes to obtain a DNA solution. 1×10⁶ cells were disseminated on a 60-mm dish and cultured overnight at 37° C. in a CO₂ environment. The cultured cells were washed about twice with 2 ml of serum-free medium. After 2 ml of serum-free medium was freshly added to the cultured cells, the total amount of the DNA solution was added to the dish, which was left to stand at 37° C. for 3 hours in a CO₂ environment. Thereafter, the medium was exchanged with an ordinary serum medium and cultivation was continued for 2 to 3 days.

As a control, a pMEHis vector with no human CaT1 gene was transfected to CHO cells.

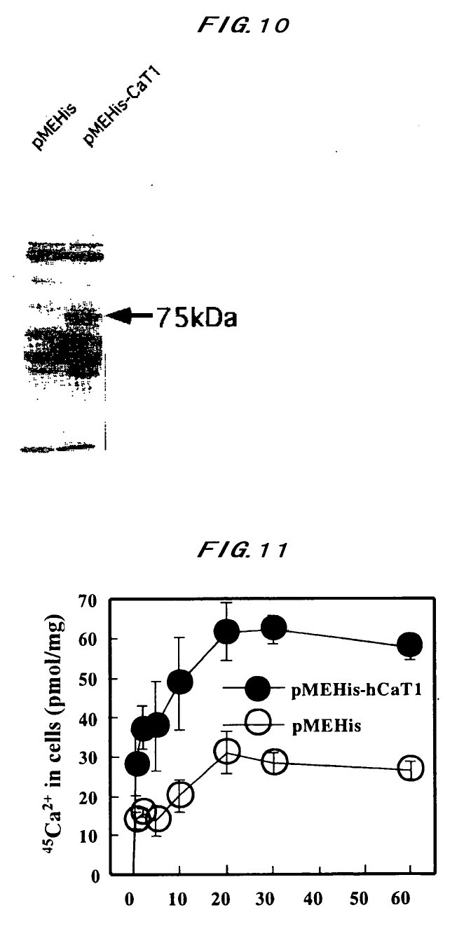

Whether or not the transfected CHO cells express the human CaT1 gene was confirmed by Western blotting using anti-hCaT1 antibody prepared in a rabbit. That is, a colony of the transformed CHO cells is selected using blasticidin and solubilized with 1% NP-40 (Nonidet P-40 surfactant, manufactured by Nacalai Tesque. Inc.). After purification with Ni resin and electrophoresis by SDS-PAGE, a construct having 1.2 Kbp of the C-end (a portion of bases 757 to 1923 of the base sequence described in SEQ ID No: 1 in the sequence listing) of the human CaT1 inserted in a pET28a vector was prepared and this was transformed to Escherichia coli BL21 to obtain fusion protein. The obtained fusion protein was used as an antigen to immunize a rabbit to prepare anti-human CaT1 antibody. The antibody was used to perform Western blotting. Results obtained are shown in FIG. 10. In FIG. 10, the right hand side shows results of the CHO cells to which pMEHis-CaT1 was transfected while the left hand side shows results of the CHO cells to which a pMEHis-vector was transfected.

As apparent from FIG. 10, a specific band of about 75 kDa (see an arrow in FIG. 10) was observed in the CHO cells transfected with pMEHis-CaT1, i.e., human-CaT1 gene-introduced cells, so that expression of the human CaT1 on a protein level could be confirmed. This indicates that the cells obtained by introducing the human CaT1 gene in the above-mentioned manner are human CaT1 gene constant expression cells.

(4) Confirmation of Calcium Uptake by Human CaT1 Constant Expression Cells

Then, whether or not the human CaT1 constant expression cells prepared in (3) above-described actually incorporate calcium was confirmed according to the procedure outlined in FIG. 7.

That is, 1×10⁵ cells/well of human CaT1 constant expression cells were cultured on a 24-well plate using a medium obtained by adding 10% FCS (fetal calf serum) was added to the above-mentioned DMEM/F12 medium at 37° C. for 5 days in a CO₂ environment. After the medium was removed and the cells were washed with PBS buffer and HBSS buffer, to each well added was HBSS buffer containing 5.28 μM of ⁴⁵ Ca²⁺, which was removed after a predetermined time (usually for 10 minutes excepting the case in which cultivation time is varied (time course is to be obtained)), the cells were solubilized with 1% triton (triton X-100), and then the cells were recovered. The amount of ⁴⁵Ca²⁺ incorporated into the cells was measured using a liquid scintillation counter. As a control, CHO cells transfected with no human CaT1 gene were also subjected to the same operations to measure the amount of ⁴⁵Ca²⁺ incorporated in the cells.

The time course of the amount of⁴⁵Ca²⁺ incorporated into the cells is shown in FIG. 11. As apparent from FIG. 11, the amount of ⁴⁵Ca²⁺ incorporated into the human CaT1 constant expression cells increased as compared with the CHO cells transfected with no human CaT1 gene.

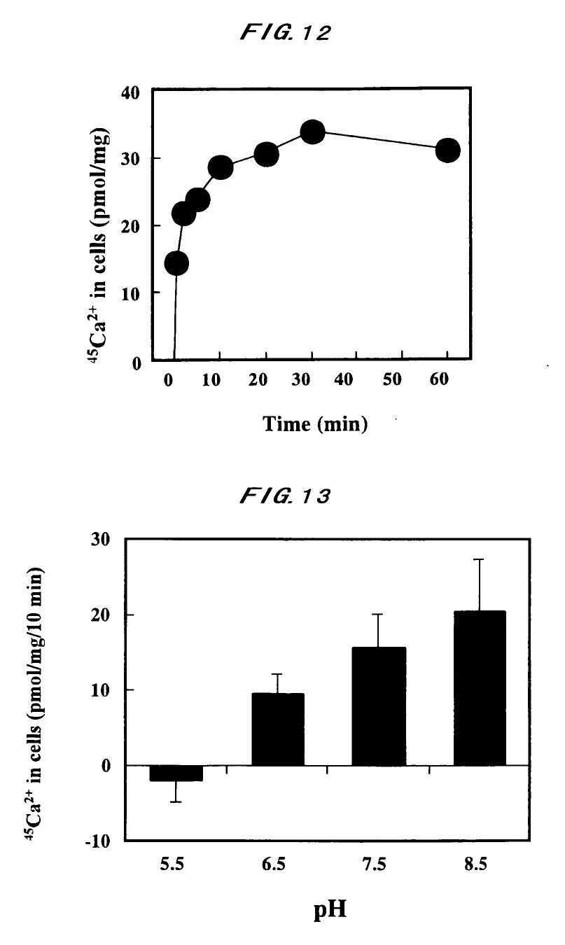

Further, the amount obtained by deducing the amount of⁴⁵Ca²⁺ incorporated into the CHO cells transfected with no human CaT1 gene from the amount of ⁴⁵Ca²⁺ incorporated into the human CaT1 constant expression cells is considered to indicate the amount of calcium that was incorporated into the cells under the participation of the human CaT1 gene. Accordingly, the time course of the amount of calcium that was incorporated into the cells under the participation of the human CaT1 gene out of the total amount of the calcium incorporated into the cells is shown in FIG. 12. FIG. 12 shows that the amount of ⁴⁵Ca²⁺ incorporated into the cells reaches a substantially constant level in 30 minutes, which clearly indicates that the uptake of calcium under the participation of the human CaT1 gene proceeds in a relatively short time and saturates.

The subsequent data on the amount of the ⁴⁵Ca²⁺ incorporated into the cells is deemed to indicate the amount of ⁴⁵Ca²⁺ incorporated into the cells under the participation of the hCaT1 as shown in FIG. 12.

(5) Influences of pH and Metal Ions on Uptake of Calcium Through hCaT1

The influences of pH and metal ions on the uptake of calcium through hCaT1 were examined to characterize hCaT1 (constant expression cells).

The influence of pH was examined by the same culture conditions and procedures as those in (4), except that the HBSS buffer containing 5.28 μM of ⁴⁵Ca²⁺ adjusted to a pH of 5.5, 6.5, 7.5 or 8.5 respectively was used. Results obtained are shown in FIG. 13. FIG. 13 indicates that the amount of ⁴⁵Ca²⁺ incorporated into the cells increases on neutral side while it is inhibited on acidic side.

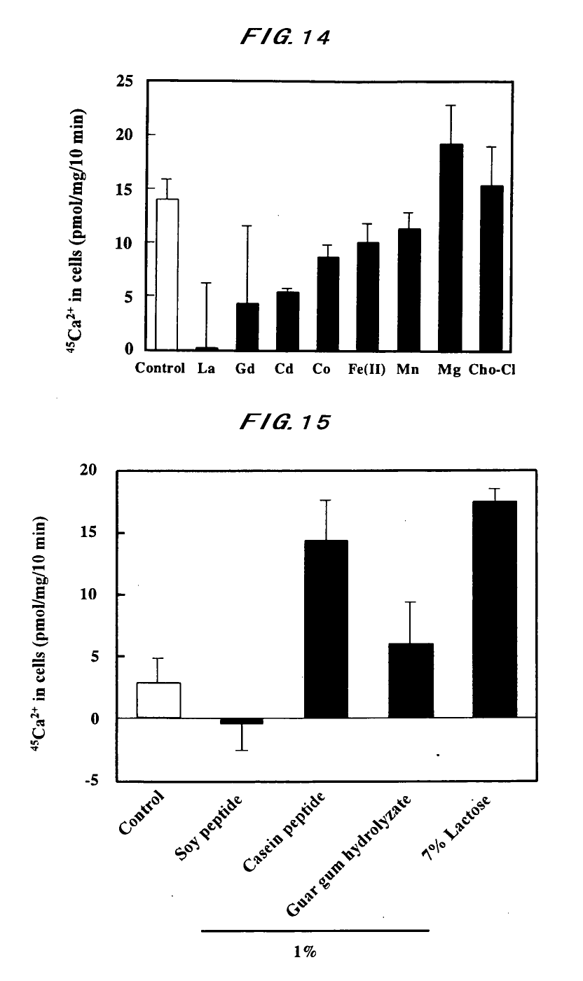

On the other hand, the effects of the metallic ion were examined by the same culture conditions and procedures as those in (4) except that 100 μM each of chloride salts La³⁺, Gd³⁺, Cd²⁺, Co²⁺, Fe²⁺, Mn²⁺, and Mg²⁺ (10 mM for Mg2+ only) and 100 μM of choline chloride (Cho-Cl) were added to a HBSS buffer containing 5.28 μM of ⁴⁵Ca²⁺. Results are shown in FIG. 14.

As apparent also from FIG. 14, La³⁺, Gd³⁺, Cd²⁺, and Co²⁺ ions each inhibit the amount of⁴⁵Ca²⁺ incorporated, while Fe²⁺, Mn²⁺, and Mg2+ show no significant difference as compared with a control, indicating that Fe²⁺, Mn²⁺, and Mg²⁺ each have no influence on the uptake of calcium.

(6) Clarification of Calcium Absorption Regulating Activity Mechanism of Food Factors that Regulate Calcium Absorption

With a view to obtaining information on the mechanism of calcium absorption regulating activity of food factors that regulate calcium absorption, the inventors of the present invention have studied the influence of the food factors on calcium uptake by the human CaT1 constant expression cells according to the procedure outlined in FIG. 8 and tried to clarify the calcium absorption regulating activity mechanism of the food factors.

In other words, in regards to a cultured human CaT1 constant expression cells evaluated by the same culture conditions and procedures as those in (4), a medium was displaced by a DMEM (—Ca) medium (Dulbecco's Modified Eagle Medium (cont.) Base Catalog No. 21068, manufactured by GIBCO™ Invitrogen Corporation: KCl 400.00 mg/l, NaCl 6400.00 mg/l, NaHCO₃ 3700.00 mg/l, NaH₂PO4.H₂O 125.00 mg/l, D-glucose 4500.00 mg/l, L-isoleucine 105.00 mg/l, L-leucine 105.00 mg/l, L-lysine.HCl 146.00 mg/l, L-tyrosin/2Na.2H₂O 104.00mg/l) containing, as the food factor, 7% lactose (special grade reagents) 1% casein enzymatic degradation products, 1% guar gum hydrolysate, and soybean enzymatic degradation products (manufactured by Fuji oil Co., Ltd.), respectively, and the amount of ⁴⁵Ca²⁺ incorporated into the cells was measured after preincubation for additional 4 hours. Results are shown in FIG. 15.

As apparent from FIG. 15, the amount of ⁴⁵Ca²⁺ incorporated into the cells increased with 7% lactose, 1% casein enzymatic degradation products, and 1% guar gum hydrolysate. On the other hand, with soybean enzymatic degradation products, the amount of ⁴⁵Ca²⁺ incorporated into the cells were contrary suppressed.

This indicates that lactose, casein, and guar gum hydrolysate stimulate the activity of the human CaT1 to promote calcium absorption while soybean enzymatic degradation products suppress the activity of the human CaT1 to inhibit calcium absorption.

### EXAMPLE 2

Influences of an enzymatic degradation product of cheese whey (CWP-D) as a food factor on calcium uptake by the human CaT1 constant expression cells obtained in Example 1 were tried to clarify presence or absence of calcium absorption regulating activity and mechanism hereof.

(1) Preparation of CWP-D by Enzyme Treatment

35% Cheese whey was prepared and 0.12% protease A (manufactured by Amano Pharmaceutical Co., Ltd.) was added to solubilize it. Then, the resultant was incubated at 37° C. for 6 hours. After the treatment, the mixture was heated at 90° C. for 10 minutes to deactivate the enzyme. After cooling, the reaction mixture was freeze-dried.

The obtained powder as an enzyme-treated sample was purified by the following procedures.

(2) Influence of CWP-D on Calcium Uptake by Human CaT1 Constant Expression Cells

The enzyme-treated sample obtained in (1) was dissolved in DMEM (—Ca) (the same as that used in Example 1(6)) so as to make a concentration of 1.0 (w/v)%. This was added to human CaT1 constant expression cells cultivated by the same culture conditions and the same procedures as those in Example 1(4) and CHO cells transfected with no human CaT1 gene, and these cells were preincubated for 4 hours. A medium contained 2.5% dialyzed (Spectra/POR MWCO:100) FCS and 2% L-glutamine solution.

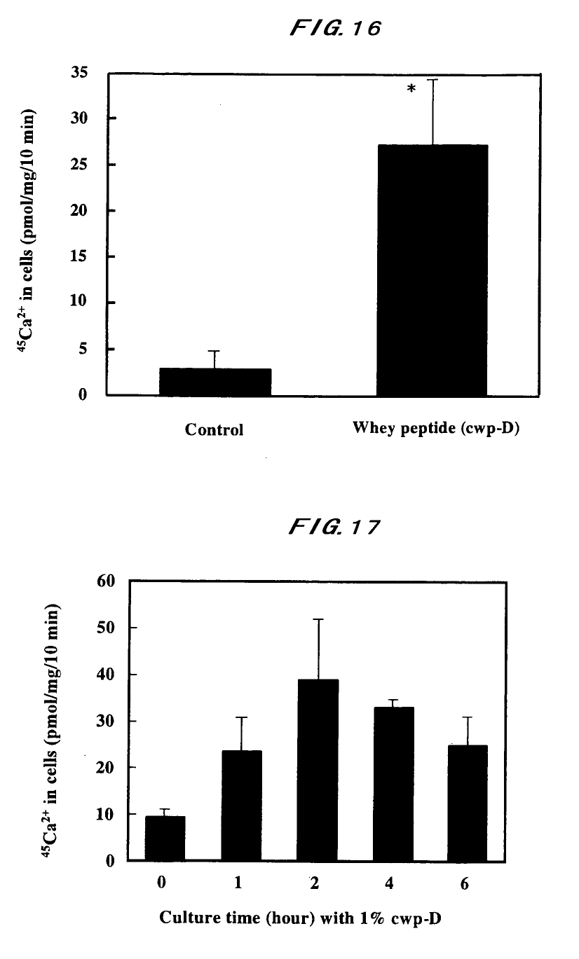

Immediately after completion of the pretreatement, the amount of ⁴⁵Ca²⁺ incorporated into the cells was measured by the same technique as used in Example 1(4). On the other hand, the same procedure was followed without addition of the enzyme-treated samples as a control. Results obtained are shown in FIG. 16.

As apparent from FIG. 16, when CWP-D was added, the amount of ⁴⁵Ca²⁺ incorporated into the cells is 8 times as high as that of the control. As a result of analysis by ANOVA, a significant difference at p<0.01 was observed between the two.

This clearly indicates that CWP-D stimulates the activity of human CaT1, thereby significantly promoting calcium absorption.

(3) Influence of Pretreatment Time on Calcium Uptake by Human CaT1 Constant Expression Cells

Influence of pretreatment time of CWP-D on ⁴⁵Ca²⁺ uptake by human CaT1 constant expression cells were examined by the same procedures as in (2), except that the pretreatment time was changed to 0 hour, 1 hour, 2 hours, 4 hours, and 6 hours in (2). Results obtained are shown in FIG. 17.

As apparent from FIG. 17, the amount of ⁴⁵Ca²⁺ incorporated into the cells increases depending on the length of the pretreatment time, saturating in 2 hours.

(4) Influence of the Concentration of CWP-D on Calcium Uptake by Human CaT1 Constant Expression Cells

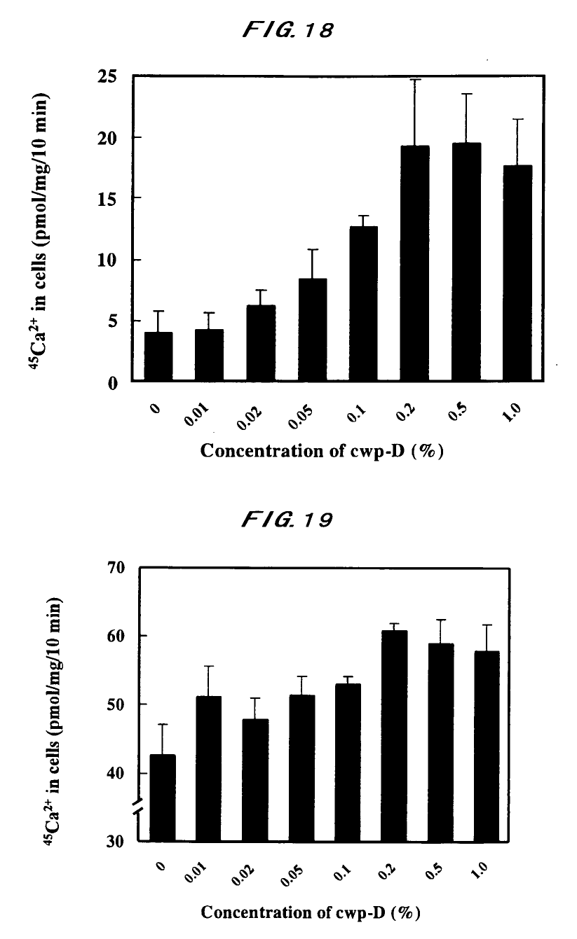

Influence of the concentration of CWP-D on ⁴⁵Ca²⁺ uptake by human CaT1 constant expression cells were examined by the same procedures as in (2), except that the concentration of the enzyme-treated sample in the medium was changed to 0 (w/v)%, 0.01 (w/v)%, 0.02 (w/v)%, 0.05 (w/v)%, 0.1 (w/v)%, 0.2 (w/v)%, 0.5 (w/v)%, or 1.0 (w/v) % in (2), and treatment was performed for each medium. Results obtained are shown in FIG. 18.

As apparent from FIG. 18, the amount of ⁴⁵Ca²⁺ incorporated into the cells increases depending on the concentration of CWP-D.

(5) Influence of CWP-D on Calcium Uptake by Caco-2 Cells

Caco-2 cell, which is one of human intestinal epithelial cells, expresses endogenous CaT1. Accordingly, influence of the concentration of the enzyme-treated sample on the uptake of ⁴⁵Ca²⁺ by Caco-2 cells were examined by the same procedures as those in (4) above except that Caco-2 cells were used instead of the human CaT1 constant expression cells. Results obtained are shown in FIG. 19.

As apparent from FIG. 19, the results clearly indicated that the amount of ⁴⁵Ca²⁺ incorporated by Caco-2 cells increases depending on the concentration of CWP-D and significantly increases at a concentration of 0.1 (w/v) % or more and the increase saturates at 0.2 (w/v)%.

(6) Influence of CWP-D on Kinetics in Calcium Uptake

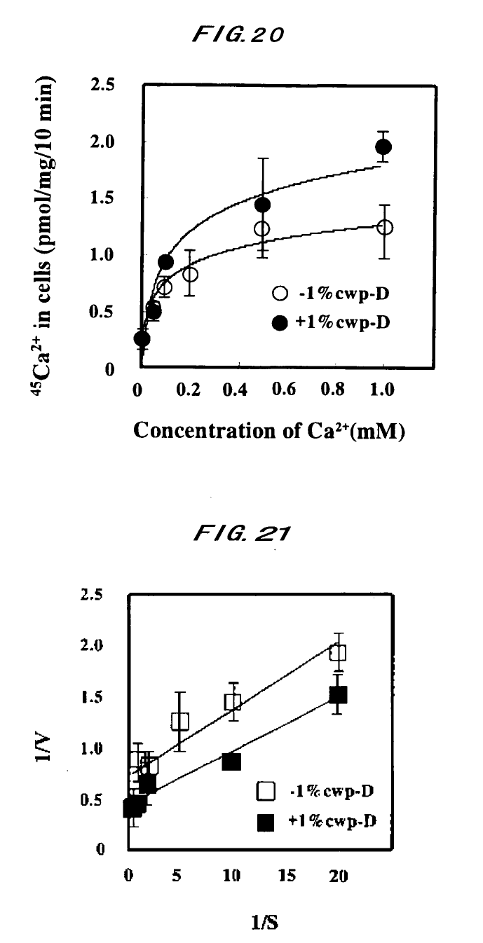

After completion of the pretreatment in (2), the concentration of CaCl₂ in the medium was varied in the range from 0.01 mM to 1.0 mM and the amount of ⁴⁵Ca²⁺ incorporated into the cells was measured during the variation (+CWP-D). On the other hand, control was treated similarly with no addition of the enzyme-treated sample. As a result, a calcium saturation curve as shown in FIG. 20 was obtained.

Based on FIG. 20, Lineweaver-Bark plotting was performed (FIG. 21), and Vmax value and Km value were calculated. The obtained Vmax value and Km value are shown in Table 1.

TABLE 1
[Influence of CWP-D on kinetics in calcium uptake]

| | Km(mM) | Vmax(pmol/mg/10 min) |
|---|---|---|
| +CWP-D | 0.019 | 1.061 |
| −CWP-D | 0.043 | 1.158 |

As apparent from Table 1, Vmax of Ca²⁺ uptake into the cells showed substantially no change between +CWP-D and −CWP-D, while a decrease in Km was observed.

This suggests that CWP-D changes the conformation of CaT1 to vary the kinetics of uptake of ⁴⁵Ca²⁺ into the cells.

(7) Isolation of Calcium Absorption Promoters Derived from CWP-D

1. Crude Purification with ODS Column

100 g of the enzyme-treated sample (CWP-D) obtained in (1) was dissolved in 300 ml of distilled water and the solution was centrifuged at 5° C. (11,000 rpm, 20 min), followed by filtration (ADVANTEC Filter Paper No. 1) to remove the fat layer. The defatted fraction was dialyzed (fraction molecular weight of 10,000, against 4 L of distilled water, three times), and the obtained external solution in the dialysis was concentrated under reduced pressure to 55.5 folds (final 200 ml). The concentrate was passed through ODS column (Wako gel 50C18, 10×100 cm), and then the adsorbed fraction was eluted with 80% ethanol. The eluate was concentrated under reduced pressure and freeze-dried to obtain a crude-purified ODS adsorbed fraction.

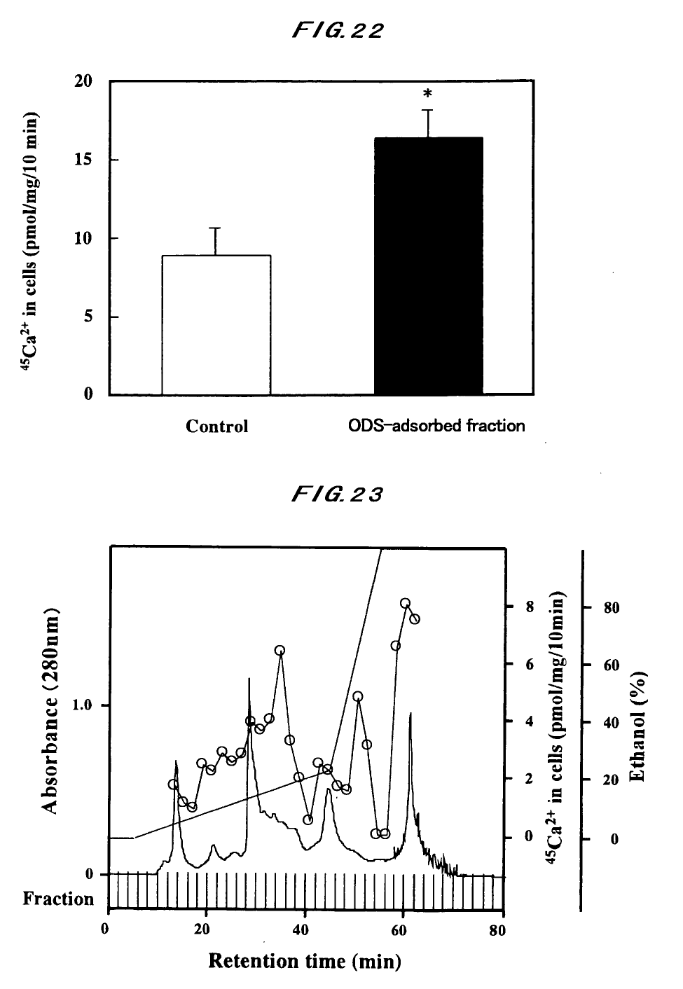

The same procedure as that in (2) was performed except that the ODS-adsorbed fraction obtained in (7)1. was used instead of the enzyme-treated sample in (2). Results obtained are shown in FIG. 22.

As apparent from FIG. 22, the amount of ⁴⁵Ca²⁺ incorporated into the human CaT1 constant expression cells is high as compared with the control when CWP-D was added. As a result of the analysis by ANOVA, a significant difference at p<0.01 was observed between the two.

This indicates that the ODS-adsorbed fraction contains a factor that promotes calcium absorbing and the following FPLC was performed to further purify the factor.

2. Reverse Phase Chromatography (FLPC)

The ODS-adsorbed fraction obtained in 1. was dissolved in distilled water so as to be at a concentration of 92.5 g/5 ml, and the obtained solution was filtered through No. 1 Filter Paper, followed by readjusting to 5 ml. The resultant was subjected to FPLC. The conditions of FPLC are as follows.

[FPLC Conditions]

Column: Wakogel 50C18 0.8×50 cm, Vt=100 ml
Sample Loop: 5 ml super loop
Detection: 280 nm
Solvent: A. H₂O (distilled water)
   B. EtOH
Fraction: 10 ml/2 min (5 ml/min)

Concentration gradient: As shown below in table 2.

TABLE 2

| Time(min) | A(%) | B(%) |
|---|---|---|
| 5 | 100 | 0 |
| 45 | 80 | 20 |
| 55 | 0 | 100 |

The ODS-absorbed fraction in 1. was previously poured in a sample loop and charged in a column sufficiently equilibrated. Sample charge, liquid supply, concentration gradient of the solvent, fraction collector and the like were automatically performed by using a computer program. Results of FPLC are shown in FIG. 23. Each of the obtained fractions was freeze-dried and then dissolved in 1 ml of distilled water and provided as an FPLC fraction in the following tests.

The amount of ⁴⁵Ca²⁺ incorporated into the cells was measured by the same procedure as that in (2) except that, instead of the enzyme-treated sample in (2), each FPLC fraction in a concentration of 0.5 mg/ml based on the human CaT1 constant expression cells was used. Results for each fraction together with the results of FPLC are shown in FIG. 23.

As apparent from FIG. 23, the amount of ⁴⁵Ca²⁺ incorporated into the cells was large in the case of the FPLC fraction having a retention time of 34 to 36 minutes.

This indicates that the FPLC fraction having a retention time of 34 to 36 minutes contains the calcium absorption promoter. Accordingly, the following FPLC was performed for further purification of the fraction.

3. Reversed Phase Chromatography (HPLC)

The FPLC fraction of which the calcium absorption increasing activity was confirmed in 2. was subjected to HPLC. The conditions of HPLC were as follows.

[HPLC Conditions]

Column: Docosil 4.6×250 mm
Sample Loop: 200 μl
Solvent: A. H₂O+0.1% TFA
   B. Acetonitorile +0.1% TFA
Detection: 214 nm
Fraction: 1 ml/min

Concentration gradient: As shown below in table 3.

TABLE 3

| Time(min) | A(%) | B(%) |
|---|---|---|
| 5 | 95 | 5 |
| 65 | 20 | 80 |

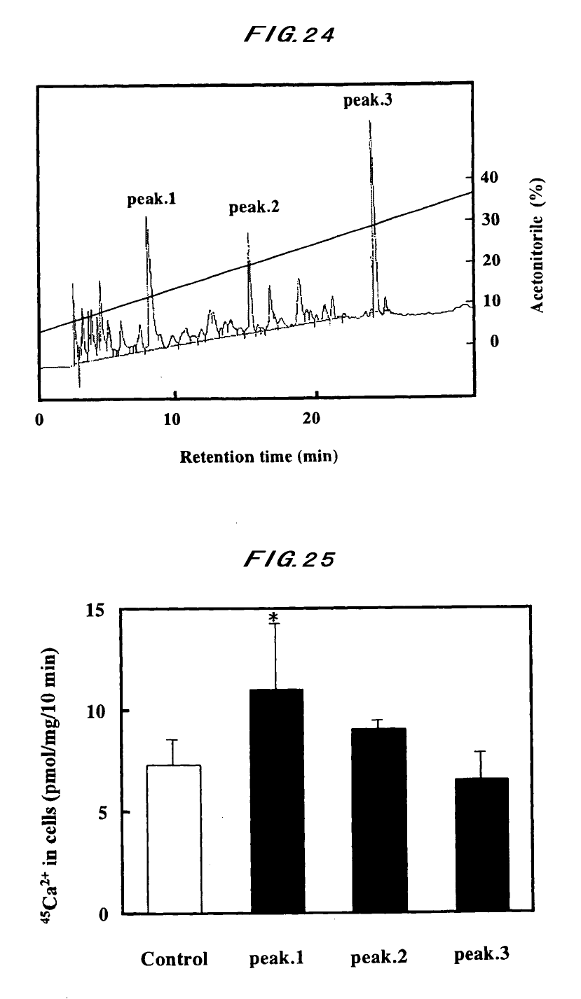

Results of HPLC are shown in FIG. 24.

As apparent from FIG. 24, major peaks (peaks 1, 2, and 3) were detected at respective positions corresponding to retention times of 8.450, 16.108, and 25.696. Accordingly, the amounts of ⁴⁵Ca²⁺ incorporated into the cells were measured by the same procedure as that in (2) except that after each peak was recovered, freeze-drying thereof were performed and that instead of the enzyme-treated sample in (2), the three recovered peaks 1, 2, and 3 were used respectively in a concentration of 0.5 mg/ml based on the human CaT1 constant expression cells. On the other hand, as a control, the same treatment without addition of the recovered peaks was performed. Results are shown in FIG. 25.

As apparent from FIG. 25, the results of analysis by ANOVA indicated that the amount of ⁴⁵Ca²⁺ incorporated into the cells when peak 1 was added increased significantly (p<0.05) as compared with other peaks and the control.

This indicates that the peak 1 contains the calcium absorption promoter.

4. Measurement of Purity of Peak 1

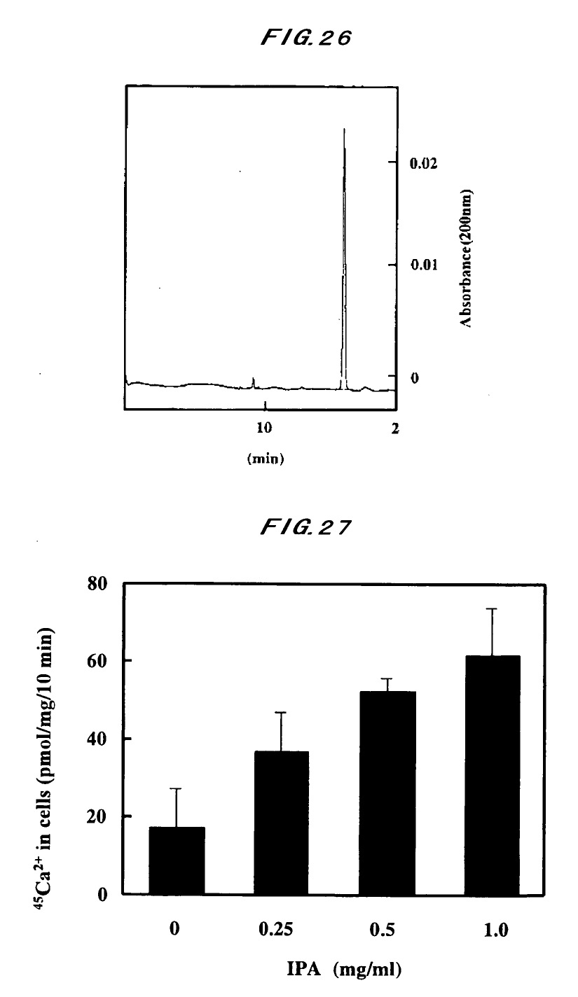

The peak 1 obtained in 3. was treated with N₂ gas and then dried. The dried residue was dissolved in phosphate buffer (pH 2.5) and the solution was passed through a 0.45-μm filter. This was subjected to capillary electrophoresis using CAPI-3000 Integrator manufactured by Otsuka Electronics to assay peptide purity. The capillary electrophoresis was performed by detection at absorbance of 200 nm under the conditions of a voltage of 15 kv and a temperature of 25° C. Results obtained are shown in FIG. 26.

As apparent from FIG. 26, only a single peak was detected, confirming that the peak 1 consists of a single substance.

5. Determination of Sequence of CWP-D

The peak 1 that was confirmed to consist of a single substance in 4. was subjected to sequence determination using a Protein Sequence System manufactured by Applied Biosystem. As a result, it has been clarified that the primary amino acid sequence of the substance contained in the peak 1 is identified to be Ile-Pro-Ala.

(8) Effects of CWP-D-derived Calcium Absorption Promoter on Calcium Uptake

A peptide having the primary amino acid sequence of the substance obtained in (7) was synthesized and the effect of the synthetic peptide IPA on the calcium uptake was examined by the following procedure.

1. Influence of Concentration of Synthetic Peptide IPA on Calcium Uptake by Human CaT1 Constant Expression Cells

The same procedure as that in (2) was performed except that instead of the enzyme-treated sample, the synthetic peptide IPA was used in concentrations of 0 mg/ml, 0.25 mg/ml, 0.5 mg/ml, and 1.0 mg/ml per cell to measure the amounts of ⁴⁵Ca²⁺ incorporated into the cells. Results obtained are shown in FIG. 27.

As apparent from FIG. 27, the amounts of ⁴Ca²⁺ incorporated into the cells significantly increased depending on the concentration of the synthetic peptide IPA.

2. Influences of Synthetic Peptide IPA, IPA Analogues and Amino Acids on Calcium Uptake by Human CaT1 Constant Expression Cells

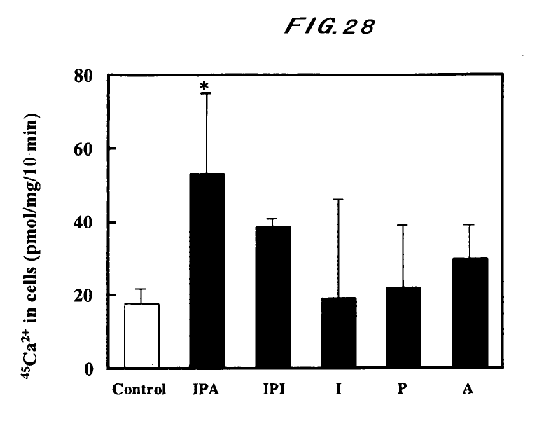

The amounts of ⁴⁵Ca²⁺ incorporated into the cells were measured by performing the same treatment as that used in (2) except that instead of the enzyme-treated sample, the synthetic peptide IPA, IPI (Ile-Pro-Ile, available from Peptide Research Institute) as an IPA analogue (analogues of the synthetic peptide), and amino acids (Ile (I), Pro (P), and Ala (A)) that constitute the synthetic peptide IPA were used in amounts of 3.0 mM, respectively. On the other hand, as a control, the same treatment was performed without adding peptides or amino acids. Results are shown in FIG. 28.

As apparent from FIG. 28, the results of the analyses by ANOVA indicates that in addition to IPI as an IPA analogue, all of Ile, Pro and Ala showed no significant increase in the amount of ⁴⁵Ca²⁺ incorporated into the cells.

The above-mentioned results demonstrate that the enzymatic degradation products of cheese whey (CWP-D) and the synthetic peptide IPA are useful as factors that promote calcium absorption.

### INDUSTRIAL APPLICABILITY

The present invention is useful to provide helpful means which enables: the genetic applications such as insertion of human CaT1 gene into an expression vector and transformation of human CaT1 gene; the clarification of the calcium absorption activity mechanism such as the presence of a factor affecting the regulation of the calcium absorption activity of human CaT1; and the confirmation and new finding of a calcium absorption regulator, particularly a factor that promotes calcium absorption.

1. A human calcium transporter 1 gene comprising a base sequence described in SEQ ID No: 1 in the sequence listing.

2. A plasmid vector comprising a human calcium transporter 1 gene according to claim 1.

3. A transformant transformed with a plasmid vector according to claim 2.

4. A method of screening a factor that regulates calcium absorption, comprising:
confirming an amount of calcium incorporated into a transformant according to claim 3.

5. A kit for screening a factor that regulates calcium absorption, comprising a transformant according to claim 3.

6. A factor that promotes calcium absorption obtained by a method of screening according to claim 4.

7. A factor that promotes calcium absorption obtained using a kit for screening according to claim 5.
